(19) United States
(12) Patent Application Publication
(10) Pub. No.: US 2006/0206011 A1
(43) Pub. Date: Sep. 14, 2006

(54) System and method for remote monitoring of multiple healthcare patients

(76) Inventors: Michael S. Higgins, Nashville, TN (US); Paul J. St. Jacques, Franklin, TN (US); Nimesh P. Patel, Nashville, TX (US)

Correspondence Address: MILES & STOCKBRIDGE PC, 1751 PINNACLE DRIVE, SUITE 500, MCLEAN, VA, 22102-3833, US

(21) Appl. No.: 11/074,004

(22) Filed: Mar. 8, 2005

(51) Int. Cl. A61B 5/00; G08B 23/00

(52) U.S. Cl. 600/300; 128/920; 340/573.1

(57) ABSTRACT

A system and method for remotely monitoring multiple healthcare patients simultaneously is described. Multiple patients in multiple locations can be monitored for a variety of healthcare conditions and indicators. Information about the monitored patients is transmitted to a local monitoring component, which can be part of a portable processing system, and can be monitored by a healthcare professional using the portable processing system at any location convenient to the professional. An adaptive algorithm dynamically determines if an alert should be generated for a patient based on historical information associated with the patient or a procedure, situational information associated with a schedule, coordination or prioritization of multiple procedures, or predictive information based on the likelihood of a future condition based on a patient's current condition, a procedure being performed, or other factors.

### FIELD OF THE INVENTION

The invention relates to providing healthcare services. More specifically, the invention relates to remotely monitoring multiple healthcare patients. The invention also has applicability within other fields where remote monitoring capabilities are desirable.

### BACKGROUND

In recent years, healthcare providers have been under increasing pressure to treat, effectively and efficiently, an ever-increasing number of patients. This is particularly true in view of the advent of managed care, which requires healthcare providers to better manage costs associated with treatment of patients. In addition to cost pressures caused by the managed care providers, healthcare providers are experiencing other growing cost pressures as well, such as rapidly rising malpractice premiums and increased drug costs.

Hospitals generally, and operating rooms (ORs) in particular, for example, are locations where efficient management of costs and resources is necessary in a managed care environment because they are centers of both high resource utilization and high revenue generation. Being centers of high resource utilization and high revenue generation, inefficiencies in hospitals and operating rooms have a potentially greater impact on profitability. Additionally, because of the greater risk often associated with such locations, healthcare workers working within those areas often have higher malpractice premiums, which can also have an impact on profitability.

Because of these and other increased cost pressures, there is a desire by many in the healthcare industry to manage costs more efficiently. One possible way to reduce operational costs, for example, is to reduce the number of healthcare practitioners in a hospital at any given time. Another possible way to reduce costs of medical procedures is to reduce the number of healthcare practitioners involved in such procedures.

In addition to reducing costs, increasing the efficiency of any given healthcare practitioner can also help manage costs. Thus, if a practitioner is able to become more efficient, such as by becoming involved in a greater number of procedures or with a greater number of patients, the cost-per-procedure or cost-per-patient for that practitioner will decrease. Because the cost of supporting that practitioner will be more efficiently allocated to a greater number of patients. Of course, as a practitioner becomes involved in a greater number of procedures or with a greater number of patients, the potential for that practitioner to make mistakes or to be unable to provide adequate attention to any one of the procedures or patients also increases.

Therefore, it would be desirable to develop a system or method capable of reducing the number of practitioners necessary in a healthcare setting, such as a hospital. Additionally, it would be desirable to develop a system or method capable of allowing a healthcare practitioner to be involved safely in a greater number of procedures or with a greater number of patients without increasing the potential for mistakes.

### SUMMARY

Accordingly, one or more embodiments of the invention provide a system and method for remote monitoring of multiple healthcare patients. According to this system and method, a healthcare practitioner is able to monitor safely a greater number of procedures or patients, which allows for increased efficiency of that healthcare practitioner. Additionally, because of the increased efficiency provided by this system and method, a reduction of the overall number of practitioners required in a healthcare setting (e.g., a hospital, an operating room suite, etc.) can be reduced. This can be facilitated, for example, by using a portable computing device that includes a local monitoring component, configured to monitor multiple patients simultaneously, and to generate alerts to the user (e.g., a healthcare practitioner) when one of the multiple patients being monitored requires attention, or when an event of interest occurs. Alternatively, the increased efficiency according to this system and method can be facilitated by a portable computing device that displays alerts generated by a similar alert generation technique performed at a location remote from the portable computing device.

For example, an embodiment of the invention provides a method, which includes monitoring data associated with multiple patients and simultaneously displays data associated with at least two of the multiple patients. A determination is made regarding whether an alert should be generated for one of the multiple of patients at least partially based on data associated with the patient, and also based on a comparison of a potential priority of the alert, which is at least partially based on the data associated with the patient, and a priority of the data being displayed. An alert is generated in substantially real-time for the patient if it is determined that an alert should be generated for the patient. The method can display such data as vital signs, video, or other information associated with the patient. The determination made by the method can also be based on the potential priority of the alert and a schedule of procedures to be performed. Additionally, or alternatively, the determination made by the method can also be based on at least one of historical information, situational information, and predictive information.

Another embodiment of the invention includes a method, which includes monitoring data associated with a patient and dynamically determining if an alert should be generated for the patient. The dynamic determination is made at least partially based on data associated with the patient, and on at least one of historical information, situational information, and predictive information. The method also includes generating an alert in substantially real-time for the patient if it is determined that an alert should be generated for the patient. The method can perform the dynamic determination based on historical data associated with a medical history of the patient or historical data for a procedure associated with that patient. The method can also perform the dynamic determination based on scheduling information or data associated with multiple patients, and can generate the alert based on the scheduling information or data, or various priorities (e.g., priorities associated with procedures, conditions, healthcare providers, etc.). The method can also use predictive information associated with the patient that is configured to predict the effect of a current condition on the probability of development of a future condition. Alerts generated according to the method can be escalated to other healthcare providers if required (e.g., if alerts are not responded to in a timely fashion).

Yet another embodiment of the invention includes an apparatus, which includes a receiver, a processor, and a display. The receiver is configured to receive transmissions from multiple monitors. The transmissions from each of the multiple monitors include data associated with a patient uniquely associated with that monitor. The processor is configured to analyze transmissions received from each monitor, and to dynamically determine if an alert for a patient associated with one of the multiple monitors should be generated based on at least one of historical data, situational data, and predictive data. The processor is also configured to generate an alert for the patient, if it is determined that an alert should be generated. The display is configured to display information associated with the transmissions received by the receiver according to instructions received from the processor. The display is also configured to cause selected information associated with the transmissions to be displayed, as well as to display any alerts generated by the processor. The processor can be configured to determine if an alert should be generated based on historical data (e.g., associated with a patient or a procedure), situational data (e.g., coordination and priorities among multiple patients or procedures), or predictive data (e.g., a possibility of a future condition based on a past condition or current condition/indicators). The display of the apparatus can be configured to display multiple views, including real-time video or vital sign information for one or more patients. The apparatus can also include or be included within a portable computing device, and the display can be configured to be viewed in a hands-free configuration by a user.

Additionally, another embodiment of the invention includes a processor-readable medium comprising code representing instructions to cause a processor to monitor data associated with multiple patients, dynamically determine, based on at least one of historical information, situational information, and predictive information, if data associated with a patient of the multiple patients are outside of a desired range, and generate an alert in substantially real-time for data, associated with a patient of the multiple patients, determined to be outside of the desired range.

Additionally, another embodiment of the invention includes a processor-readable medium comprising code representing instructions to cause a processor to monitor data associated with a patient, and adaptively determine if an alert should be created for the patient at least partially based on patient-specific inputs. If an alert is created, the code representing instructions cause a processor to determine if the created alert should be generated at least partially based on a comparison of the created alert and a historical input, and generate the created alert if it is determined that the created alert should be generated. For example, a reactive alert engine can receive multiple patient-specific inputs (e.g., physiologic parameters, patient context, procedure, care process, time parameters, etc.). A comparator engine can optionally use this information along with historical inputs (e.g., similar condition parameters, similar patient parameters, etc.) to determine if an alert generator should be instructed to generate an alert to a healthcare provider.

Further features of the invention, and the advantages offered thereby, are explained in greater detail hereinafter with reference to specific embodiments described below and illustrated in the accompanying drawings, wherein like elements are indicated using like reference designators.

### BRIEF DESCRIPTION OF THE DRAWINGS

FIG. 1 is a block diagram of a remote monitoring system, according to an embodiment of the invention.

FIG. 2 is a block diagram of a local monitoring component, according to an embodiment of the invention.

FIG. 3 is a block diagram of a remote monitoring device, according to an embodiment of the invention.

FIG. 4 is a flow diagram of an alert generation technique, according to an embodiment of the invention.

FIG. 5 is a flow diagram of an alert determination technique, according to an embodiment of the invention.

FIG. 6 is a flow diagram of an alert generation technique, according to an embodiment of the invention.

FIG. 7 is a block diagram of a data analysis system, according to an embodiment of the invention.

FIG. 8 is a block diagram of a display showing vital signs of multiple patients, according to an embodiment of the invention.

FIG. 9 is a block diagram of a display showing an alert generated by an alert generation system, according to an embodiment of the invention.

FIG. 10 is a screen shot of a display showing video of multiple patients during procedures, according to an embodiment of the invention.

FIG. 11 is a screen shot of a display showing various information of a single patient, according to an embodiment of the invention.

FIG. 12 is a screen shot of an alert generated by an alert generation system, according to an embodiment of the invention.

FIG. 13 is a screen shot of a multi-patient healthcare schedule, according to an embodiment of the invention.

### DETAILED DESCRIPTION

According to one or more embodiments of the invention, a system and method for remote monitoring of multiple healthcare patients are provided. An embodiment of the invention allows a healthcare provider to use a portable computing device to remotely communicate with one or more telemetry units located remotely from the portable computing device and collocated with a healthcare patient. These telemetry devices can transmit information about their associated patient to the portable device either directly or indirectly. For example, the telemetry devices, or remote monitoring devices, can include various vital sign monitors configured to transmit vital sign information of an associated patient to the portable computing device, or local monitoring component. Additionally, or alternatively, the telemetry devices can include or use other components, such as a video capture device, for example, configured to transmit video information to the portable computing device. The telemetry devices and related components can transmit information to the portable computing device by way of, for example, a wireless network, or other suitable means. When used in a wireless networking environment, the telemetry devices can also be portable, such that they are located with a patient as the patient moves throughout a care facility.

A healthcare professional or practitioner can use the portable computing device to simultaneously oversee healthcare procedures (e.g., operations, etc.) associated with multiple patients. For example, vital signs of multiple patients undergoing various medical procedures can be simultaneously viewed by a healthcare practitioner using the portable computing device, which assembles information provided by multiple telemetry devices (also referred to as remote monitoring devices). In this manner, a healthcare practitioner can use a single, portable unit wherever that practitioner is currently located to view information about multiple patients simultaneously. Additionally, where other components, such as video capture devices or the like are used at a patient location, information from those components, such as a live video feed, for example, can be displayed to the healthcare practitioner via the portable computing device. In addition to vital sign information and video capture information, other information associated with multiple patients can be transmitted to the healthcare practitioner via the portable computing device. Additionally, other relevant information, such as scheduling information, medical history information, procedure-related information, or the like, can be provided to the healthcare practitioner by way of the portable computing device.

Various processing tasks, according to one or more embodiments off the invention, can be carried out in a distributed manner by multiple devices (e.g., portable computing devices, local monitoring components, remote monitoring devices, etc.), or in a more centralized manner, using one or more devices (e.g., servers, storage devices, processor devices, etc.) centrally located within a network. For example, either the portable computing device itself, or another processor device in communication with the portable computing device (e.g., via a network connection), can monitor multiple feeds from multiple remote monitoring devices.

Additionally, the data received from multiple remote monitoring devices can be monitored for potential problems or other information that should be brought to the attention of the healthcare practitioner either locally within the portable device or centrally at a network location. When such information becomes available, or when an event occurs of which a healthcare practitioner should be aware, a dynamic determination of whether or not to generate an alert directed to that healthcare practitioner can be made. In determining whether or not to generate an alert directed to a healthcare practitioner, multiple types of information can be taken into consideration, and embodiments of the invention can adaptively generate alerts based on such information, or other considerations. For example, historical information, situational information, and predictive information can be taken into account to determine if an alert should be generated.

Alerts can be generated based on conditions or the occurrence of events of interest. For example, alerts can be generated when a patient requires attention from a healthcare provider. Additionally, alerts can be generated at important times and events. For example, alerts can be presented upon arrival of a patient to a preoperative area or operating room or at important times, such as surgical incision or the end of a procedure or operation.

Once alerts are generated, templates that provide care protocols can be accessed by a healthcare provider receiving the alert, according to one or more embodiments of the invention. For example, when a clinician receives a cardiology-related alert, that clinician can access one or more cardiology-related care protocols to assist the clinician in properly responding to the alert condition. These care protocols can be pre-programmed and customized according to policies and procedures of a healthcare facility, or according to industry standard best practices. In addition to providing care protocols, the templates can also provide other relevant information for treating the alert condition. For example, various links to relevant literature and studies can be linked to from the care protocols, such that the clinician has ready access to such information.

By using a local monitoring component, such as a portable computing device, a healthcare practitioner can potentially oversee treatment of many more procedures and patients than would otherwise be possible, thereby increasing the practitioner's efficiency. For example, an anesthesiologist using such a portable computing device can potentially oversee administration and maintenance of anesthesia to multiple patients undergoing simultaneous or overlapping procedures. Specifically, because real-time vital sign information and/or video of a patient can be viewed by the anesthesiologist, and because alerts of potential problems associated with a patient's vital signs or administration of anesthesia to a patient can be immediately and automatically brought to the attention of the anesthesiologist, he can potentially oversee many more patients or procedures than might otherwise be possible if his presence were required at each of the procedures, or if he were required to leave a central monitoring station periodically.

According to one or more embodiments of the invention, a system and method (e.g., which can be implemented using software) configured to allow mobile management of an operation room environment or a suite of operation rooms is provided. This system and method allows a healthcare provider or clinician working in specific location within a geographic area, such as an operating room suite (e.g., a group of 20-30 ORs) or a single hospital (e.g., one or more OR suites) to be continually aware of events in other locations within or outside of the geographic area. The ability to manage such an operation room environment is particularly useful for certain physicians and other healthcare providers, such as anesthesiologists, who are typically responsible for a group of ongoing operations or procedures (e.g., 2-4 procedures), and a group of patients (e.g., 4-8 patients) in the preoperative and postoperative areas associated with the geographical area within which the clinician is working. Using one or more embodiments of the invention, the clinician's work and need for access to information is mobile and may occur in numerous locations throughout the suite, which is facilitated according to embodiments of the invention that make use of a portable computing device.

Another aspect of one or more embodiments of the invention is the capability to monitor patients remotely using a mobile device. This is advantageous over traditional patient monitoring techniques that require the clinician to view patient data from a fixed workstation either at a patient location (e.g., within an OR), or at a central location (e.g., at a nursing station). For example, mobile patient monitoring capabilities can bring patient data to a physician or other healthcare provider via a wireless computer link (e.g., to a wearable computer and/or a heads-up display). Because data associated with multiple patients, as well as additional information (e.g., historical information, situational information, predictive information, etc.) can be integrated into a single application, a clinician is able to select and view data associated with multiple patients (e.g., using an attached pointing or cursor-control device). Because information is available to a clinician via a portable device (e.g., a wearable computer and/or heads-up display, etc.), the clinician does not have to change locations in order to review the data, and decisions can be made as data are reviewed wherever the clinician is located, thereby increasing the speed and efficiency with which the clinician can operate.

Information associated with multiple patients can be simultaneously viewed by a healthcare provider on a single screen. For example, multiple video feeds showing video of multiple (e.g., four or more), simultaneous procedures can be viewed simultaneously, and controlled remotely by the healthcare provider. For example, the healthcare provider can control video size and camera actions (e.g., pan, tilt, zoom, etc.). Additionally, other information from multiple patients, such as vital signs, for example, can be viewed simultaneously, or such information can be included in a display showing video feeds for each patient.

According to one or more embodiments of the invention, the systems, methods, and various techniques described herein can be integrated with existing patient management software by way of application programming interfaces (APIs) to such management software or APIs of one or more embodiments of the invention. For example, existing OR scheduling systems and/or progress systems can be incorporated with one or more of the various embodiments described herein. For example, one existing management software package that can be integrated with one or more of the embodiments of the invention is the Vanderbilt Perioperative Information Management System (VPIMS). Additionally, other systems or software packages can be integrated with one or more embodiments of the invention. For example, anesthesiology charting software (e.g., VPIMS GasChart) can be integrated with one or more embodiments of the invention, allowing data entered by an individual local to the patient (e.g., an anesthesia resident, a certified registered nurse anesthetists or CRNA, etc.).

In addition to interfacing with various existing software packages, one or more embodiments of the invention can interface with information stored either locally to (e.g., via an intranet) or remotely from (e.g., via the Internet) the various local monitoring components. For example, if patient or procedure historical information is stored by the healthcare facility, that information can be obtained via a local intranet and used according to one or more embodiments of the invention. If such patient or procedure historical information is stored (e.g., by a health insurer, healthcare organization, professional organization, etc.) in a location accessible by multiple healthcare facilities, then that information, such as a storage device accessible via the Internet or other inter-facility network, then that information can be obtained and used according to one or more embodiments of the invention via that network.

As used herein, the terms “historical information” and “historical data” can include medical or health information of a patient prior to a current procedure, information associated with prior procedures, or other previously obtained information that might be of interest in determining whether an alert should be generated concerning a patient.

As used herein, the terms “situational information” and “situational data” include information or data about situations, such as scheduling information, currently pending alerts, current procedures, prioritization of alerts or alert levels, prioritization of conditions, notification order for healthcare practitioners, or other similar situational information.

As used herein, the terms “predictive information” and “predictive data” include data that, either alone or in combination with other data, indicate a likelihood of a future condition, problem, or event of interest. For example, certain combinations of vital signs may indicate a high potential for future development of another condition, in which case those vital signs can be used as “predictive data” or “predictive information” to predict the likelihood of the other condition. Similarly, knowledge of the interaction between certain vital signs or between vital signs and other data (e.g., specific procedures, etc.) can be used as predictive information to predict a likelihood of the future occurrence of a condition, a problem, or an event of interest for a healthcare practitioner.

The terms “healthcare practitioner,” “healthcare professional,” “healthcare provider,” “healthcare worker,” and “clinician” are used interchangeably herein and are intended to be synonymous. As used herein, each of those terms is intended to include any healthcare personnel that can benefit from using the various systems and methods described herein, or can generally benefit from using a local monitoring component, such as a portable computing device, to simultaneously monitor multiple remote locations.

Parameters associated with a patient that can be monitored by way of one or more embodiments of the invention, include vital signs, laboratory data, and other measured parameters. For the sake of convenience, the term “vital signs” is used to refer to each type of parameter that can be monitored. Some vital signs that can be monitored according to one or more embodiments include: heart rate (HR), systolic blood pressure (Sys BP), diastolic blood pressure (Dia BP), mean blood pressure (Mean BP), respiration or respiratory rate (RR), tidal volume (TV), oxygen saturation percentage (SaO2 (%)), and temperature (Temp (C)).

Some types of laboratory data that can be monitored according to one or more embodiments include: acidity (pH), arterial partial pressure of carbon dioxide (pCO2), arterial partial pressure of oxygen (pO2), base excess (BE), packed cell volume (PCV), Glucose levels, ionized calcium (iCal), concentrations or amounts of lactic acid, calcium (Cal), potassium (K+), prothrombin time (PT), partial thromboplastin time (PTT), platelets (Plts (thou)), hemoglobin (Hgb), and magnesium (Mag).

Some other parameters that can be monitored according to one or more embodiments of the invention include: peak inspiratory pressure (PIP), central venous pressure (CVP), pulmonary artery pressure (PAP), pulmonary capillary wedge pressure (PCWP), cardiac output (CO), cardiac index (CI), electrocardiogram (ECG or EKG), train of four (TOF), concentration of desfulurane (Des (%)), concentration of isoflurane (Iso (%)), concentration of sevoflurane (Sevo (%)), concentration of halothane (Hal (%)), oxygen flow rate (O2 (l/m)), air flow rate (Air (l/m)), nitrous oxide flow rate (N2O (l/m)), ventilator mode (Mode), end tidal nitrous oxide concentration (Et N20 (%)), inspired fraction of oxygen (Fi O2), end tidal anesthetic agent (Et Agent (%)), end tidal carbon dioxide concentration (Et CO2 (mmHg)), helium flow rate (He (l/m)), inspired oxygen concentration (iO2 (%)), inspired anesthetic agent concentration (iAgent (%)), end tidal concentration of isoflurane (Et Iso (%)), end tidal concentration of desflurane (Et Des (%)), end tidal concentration of sevoflurane (Et Sevo (%)), end tidal concentration of halothane (Et Hal (%)), end diastolic volume index (EDVI), non-invasive cardiac output (NICO), mixed venous oxygen saturation (mv Sat), rectal temperature (T rect), nasal temperature (T nasal), skin temperature (T skin), intra-cranial pressure (ICP (mmHg)), bispectral index (BIS), peep (PEEP), helium flow rate (He (l/m)), halothane concentration (Hal (%)), S-T segment analysis (1) (ST1), and S-T segment analysis (2) (ST2).

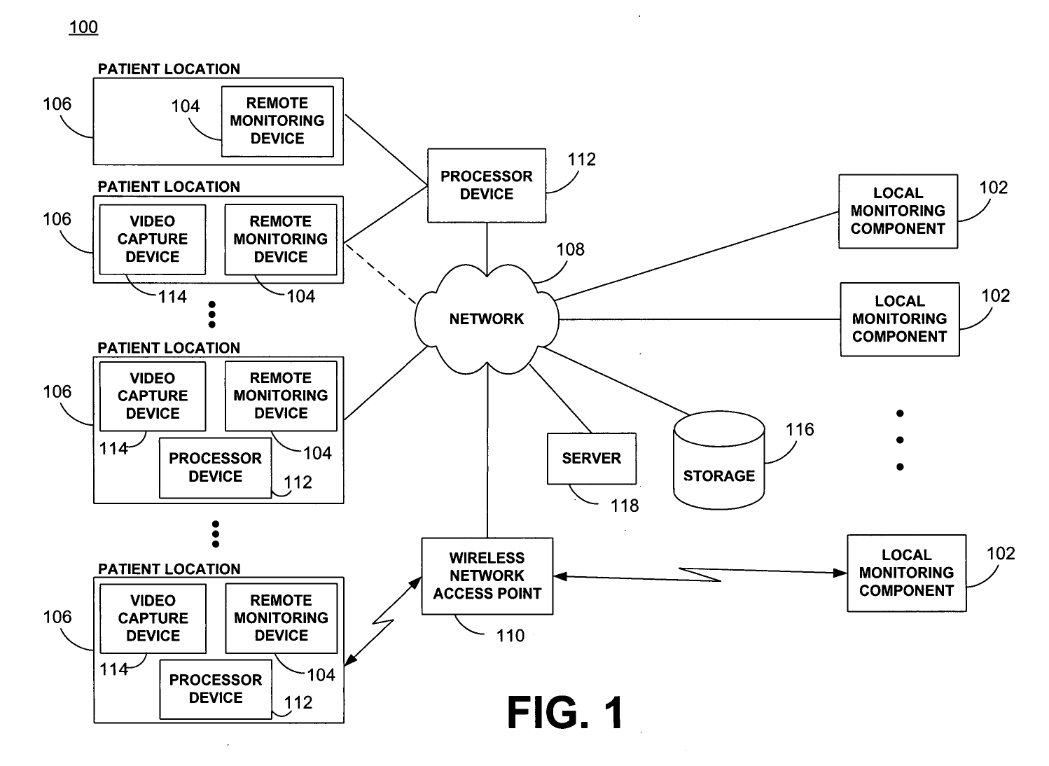

FIG. 1 is a block diagram of a remote monitoring system 100, according to an embodiment of the invention. It should be understood that, although the system 100 described in connection with FIG. 1 is described below in connection with monitoring patients in a healthcare environment, this system 100 can be modified to monitor areas of interest within other operational environments. For example, instead of the monitoring locations being patient locations in a healthcare environment, as described in detail below, the system 100 described in connection with FIG. 1 can be modified to monitor locations of interest within a security context, an inventory management environment, or other environments where the capability for a single individual or small group of individuals to remotely monitoring of multiple locations is desirable. Other uses of the system 100 shown in FIG. 1 within the scope of the invention can also be realized by modifying the system 100 in other manners as desired to accomplish objectives associated with those uses.

The remote patient monitoring system 100 shown in FIG. 1 includes one or more local monitoring components 102, which are used by a healthcare professional to monitor data associated with one or more patients. The data or other information associated with the one or more patients that is monitored via the local monitoring components 102 is received, either directly or indirectly, from one or more remote monitoring devices 104, each of which is located at a patient location 106 (or a monitoring location 106 in other monitoring environments). Examples of patient locations can include, for example, and operating room (OR) area, a pre-operative area, a triage area, a post-operative area, a recovery area, and so forth. Additionally, when a remote monitoring device 104 is portable (e.g., the remote monitoring device 104 communicates using a wireless communications capability), the patient location 106 may change with the position of the patient being monitored by the device.

Although the remote monitoring devices 104 are referred to as “remote” because they are remote from the local monitoring components 102, they can also be located within close proximity to the local monitoring component in some circumstances. For example, this is especially true in cases where the local monitoring component 102 includes or is included within a portable computing device used by a healthcare provider, which is (at least temporarily) located within the same patient location 106 as the remote monitoring device 104.

Information, such as vital signs, or other data for a patient within each patient location 106 is gathered by the remote monitoring device 104, and is transmitted to one or more local monitoring components 102 via a network 108. The network 108 can be, for example, any network suitable for transmitting such information (e.g., the Internet, a local area network (LAN), a wide area network (WAN), token ring, etc.) using one or more suitable communications protocols (e.g., TCP/IP, SPX/IPX, NetBEUI, NetBIOS, HL7, etc.). As illustrated in FIG. 1, data can be transmitted by the remote monitoring device 104 to the network 108 either directly, or by way of various devices in communication with the network 108.

In cases where the remote monitoring device 104 includes wireless communications capability, for example, data can be transmitted to the network 108 by way of a wireless network access point 110, which can make use of one or more wireless transmission protocols (e.g., infrared, Bluetooth, IEEE 802.11, 802.15, or 802.16 standards, etc.) to communicate between wireless devices. The wireless access point 110 can also act as a gateway for the remote monitoring device 104 to the network 108, via which the remote monitoring device 104 can transmit data to one or more local monitoring components 102. Similarly, other devices can act as gateways to the network 108 for the remote monitoring device 104, such as a processor device 112, or other similar computing device. A processor device 112 used as a gateway to the network 108 can provide, for example, firewall or other capabilities. Alternatively, information can be directly transmitted from the remote monitoring device 104 by way of a wireless network access point 110 to a local monitoring component or components 102, without the need for communication via a network 108.

At each of the patient locations 106, various devices can be used for obtaining information associated with a patient at that location 106. For example, a remote monitoring device 104, such as a vital sign monitor, for example, can be used alone at a patient location 106 to obtain and transmit vital sign information about a patient to a local monitoring component 102 via the network 108. In addition to a remote monitoring device 104, other devices, such as a video capture device 114, can be used to obtain and transmit additional information about a patient, such as real-time video footage of the patient location 106. This information can be transmitted via the network 108 or a wireless network access point 110 to one or more local monitoring components 102.

Additionally, or alternatively, any device at a patient location 106, such as the video capture device 114 and the remote monitoring device 104, can transmit data (e.g., video data, vital sign information, etc.) to a storage device 116 connected to the network 108. This information can be stored for later use by devices connected to the network 108, such as the processor device 112, a server 118, or one or more local monitoring components 102. Information stored within the storage device 116 can be accessed and distributed by the server 118 via the network 108. The information stored within the storage component 116 can, therefore, be requested by a local monitoring component 102, or by a server 118, which can then transmit the information to a device connected to the network 108, such as the processor device 112 or one or more local monitoring components 102.

Although only one server 118 is shown in FIG. 1, the Network 108 can use one or more servers 118 to provide various functional capabilities to the network 108 and devices connected thereto. For example, an interface server 118 can be used to provide the interface used by a local monitoring component 102, or other device connected to the network 108. A database server 118 can be provided to access data stored on the storage device 116, or within databases stored by other devices within the network 108. Additionally, or alternatively, in a centralized computing embodiment, a terminal server 118 can be provide to support thin client functionality on the local monitoring components 102, or other devices connected to the network 108. The server 118, and other devices connected to the network 108 can use a variety of suitable operating systems, such as Unix, Linux, Citrix, and various Windows operating systems (e.g., Windows XP, Windows CE, Windows 2000, Windows XP Mobile, Windows Terminal Server, etc.) available from Microsoft Corp. of Redmond, Wash.

Other components can also be located at each patient location 106, such as a processor device 112. The processor device 112 can include a microprocessor, and can, for example, perform processing on patient data associated with the patient at a patient location 106, video footage obtained by the video capture device 114, vital signs or other patient data acquired by the remote monitoring device 104, or the like. A processor device 112 at the patient locations 106 can perform pre-processing of the information to be communicated via the network 108 to the local monitoring component 102, if desired, thereby preparing the data for use by the local monitoring component 102. Such pre-processing can be used, for example, to alleviate processing burdens of a local monitoring component 102, which may have limited processing capabilities, especially if the local monitoring component includes or is included within a portable processing device.

A healthcare provider using a local monitoring component 102 as part of a wearable computer, for example, can access all of the data from the patient location 106 via the network 108 or a wireless network access point 110. This data can be either addressed to the local monitoring component 102 of the healthcare provider, or it can be specifically requested by the healthcare provider via the local monitoring component 102. Using the local monitoring component 102, a healthcare provider can monitor patients at multiple patient locations 106 simultaneously, despite the fact that those locations may be remotely located from the local monitoring component 102, and possibly remote from one another. A healthcare provider can also access information stored in a storage device 116, such as information transmitted from the patient location 106 and stored, medical records or other historical information, scheduling information associated with the healthcare environment within which the system is being used, or predictive information relevant to one or more patients being monitored. A processor, either local to the local monitoring component 102, or otherwise accessible to the local monitoring component 102 or other devices via the network 108 (e.g., the a processor of the processor device 112, or another processor accessible by the server 118) can be used to monitor all patient data from each patient location 106, and to determine whether or not an alert should be generated and transmitted to one or more local monitoring components 102, as described in greater detail below.

Other data capture devices can also be used at a patient location 106, such as an audio capture device (not shown), which can be part of a video capture device 114, part of a remote monitoring device 104, or can be independent. An audio capture device can be, for example, used to obtain auditory information associated with a patient, such as respiration or cardiologic information (e.g., via auscultation, etc.).

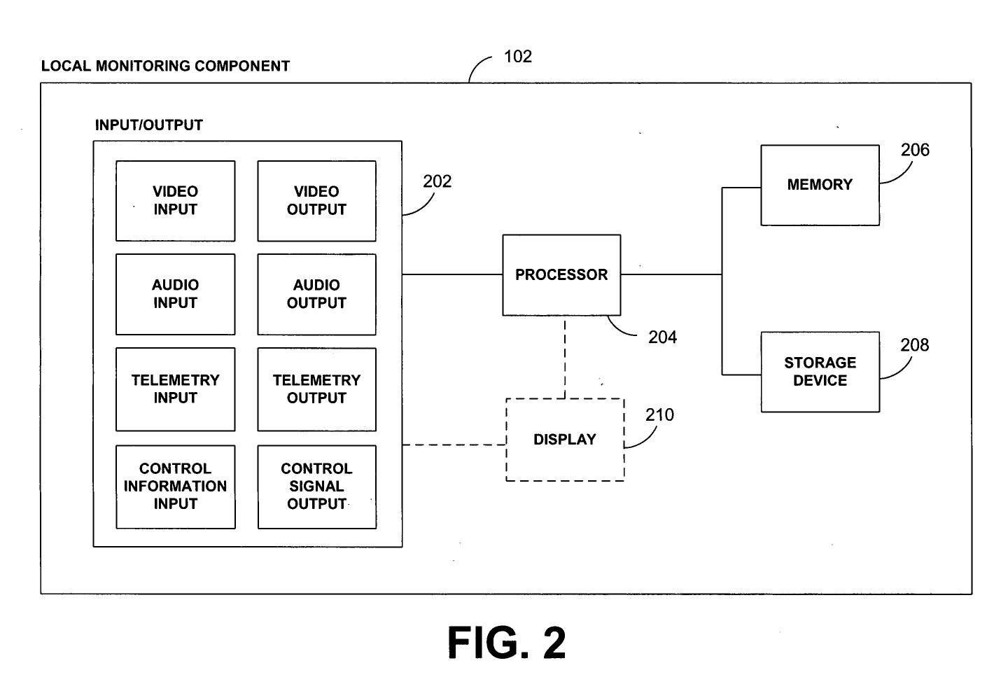

FIG. 2 is a block diagram of a local monitoring component 102, according to an embodiment of the invention. The local monitoring component 102 shown in FIG. 2 is a detailed example of a local monitoring component 102 that can be used in the system 100 shown in FIG. 1. The local monitoring component 102 can include or be included within a portable computing device, such as a personal digital assistant (PDA), a laptop computer, a tablet computer, or a wearable computer.

Examples of wearable computers that can be used with one or more embodiments of the invention (e.g., integrated with a local monitoring component or as a portable computing device) can be found in the following patents, each of which is hereby incorporated by reference in its entirety: U.S. Pat. No. 5,285,398 to Janik; U.S. Pat. No. 5,491,651 to Janik; U.S. Pat. No. 5,555,490 to Carroll; U.S. Pat. No. 5,572,401 to Carroll; U.S. Pat. No. 5,581,492 to Janik; U.S. Pat. No. 5,844,824 to Newman, et al.; U.S. Pat. No. 6,047,301 to Bjorklund, et al.; U.S. Pat. No. 6,108,197 to Janik; U.S. Pat. No. 6,157,533 to Sallam, et al.; U.S. Pat. No. 6,167,413 to Daley; U.S. Pat. No. 6,336,126 to Bjorklund, et al.; U.S. Pat. No. 6,421,232 to Sallam; U.S. Pat. No. 6,522,531 to Quintanna, et al.; U.S. Pat. No. 6,725,282 to Grzybowski, et al.; U.S. Pat. No. 6,769,038 to Grzybowski, et al.; and U.S. Pat. No. 6,798,391 to Peterson.

The local monitoring component 102 includes an input/output (I/O) component 202, which communicates with a processor 204. The processor 204 used by the local monitoring component 202 can be any suitable, commercially available microprocessor capable of performing general processing tasks, or can be an application-specific processor specifically designed to perform processing required by the local monitoring component 102. The I/O component 202 can include a variety of different I/O components, used to receive input and transmit output to and from the local monitoring component 102. Each of the I/O components of the I/O component 202 can communicate using any standard wired or wireless communications protocols.

For example, the I/O component 202 of the local monitoring component 102 can include various inputs, such as a video input, an audio input, a telemetry input, and a control information input. The video input can, for example, receive video information from a video capture device 114 at a patient location 106, or a storage device 116 (shown in FIG. 1). Similarly, an audio input can be used to receive audio input, either locally to the local monitoring component 102, or remotely from a patient location 106 (shown in FIG. 1). A telemetry input can receive data transmitted to the local monitoring component 102 via a network 108 by a remote monitoring device 104. This telemetry input can, for example, receive such telemetry as vital signs of a patient at a patient location 106, drug administration information at the patient location 106, or other patient parameters. The control information input can be used to receive control information, such as signals used to control the local monitoring component, which can be entered locally to that component by a user, or received via a network 108 (shown in FIG. 1). For example, a user desiring to change a view displayed by a display 210 connected to (or optionally part of) the local monitoring component 102 can provide control information to the local monitoring component (e.g., by way of a keyboard, one or more buttons, a cursor-control device, etc.), which can be received by the control information input.

The I/O component 202 of the local monitoring component 102 can also include output components configured to output various data and information from the local monitoring component 102. For example, the I/O component 202 of the local monitoring component 102 can include a video output, an audio output, a telemetry output, and a control signal output. The video output can be used, for example, to control a display 210, which can optionally be included within the local monitoring component 102. Although not illustrated in FIG. 2, a display 210 can also be external to the local monitoring component 102, and controlled by the video output of the I/O component 202 of the local monitoring component 102. In a similar manner, the audio output can control speakers, either included as part of the local monitoring component 102, or external thereto. The video output can be configured to display information received from the patient locations 106, including patient data (e.g., vital signs) and video information.

The audio output can be used, for example, to output audio feedback to a user of the local monitoring component 102. Additionally, the audio output can be used by a healthcare provider to listen to audio information conveyed to the local monitoring component 102 (received via the audio input) from a patient location 106. For example, the remote monitoring device 104 (shown in FIG. 1) or an independent audio capture component (not shown) can be configured to receive audio information from a patient, such as auscultations of the heart of a patient at the corresponding patient location 106.

The I/O component 202 of the monitoring component 102 can also include a telemetry output configured to output telemetry information received from one or more remote monitoring devices 104 at a patient location 106. For example, such information can be transmitted by the local monitoring component 102, either in whole or in part, to be stored by the storage device 116 (shown in FIG. 1). Thus, as with the video output and the audio output, the telemetry output can be used by a healthcare provider to transmit information desired to be stored (e.g., for clinical purposes or the like) to a storage device 116 connected to the network 108 or to other devices connected to the network 108 that are configured to use such information.

The I/O component 202 of the local monitoring component 102 can also include a control signal output, which can be used to output control signals configured to control the remote monitoring device 104, a processor device 112 connected to the network 108, a video capture device 114, a storage device 116, or other devices connected to the network 108. For example, if specific information is required from the storage device 116, the control signal output of the I/O component 202 of the local monitoring component 102 can be used to send a control signal configured to retrieve that information from the storage device 116. Additionally, when the control signal output is used to provide control signals to a video capture device 114, it can provide control signals configured to remotely cause the video capture device to change an image size, pan, tilt, zoom, and so forth.

The processor 204 controls the operation of the various I/O components 202 of the local monitoring component 102, as well as controlling other components and devices within the local monitoring component 102. For example, the processor 204 can be used to control a memory device 206, and a storage device 208 internal to the local monitoring component 102. Additionally, if a display 210 is included as part of the local monitoring component 102, the processor 204 can control the display 210 directly or via the video output of the I/O component 202.

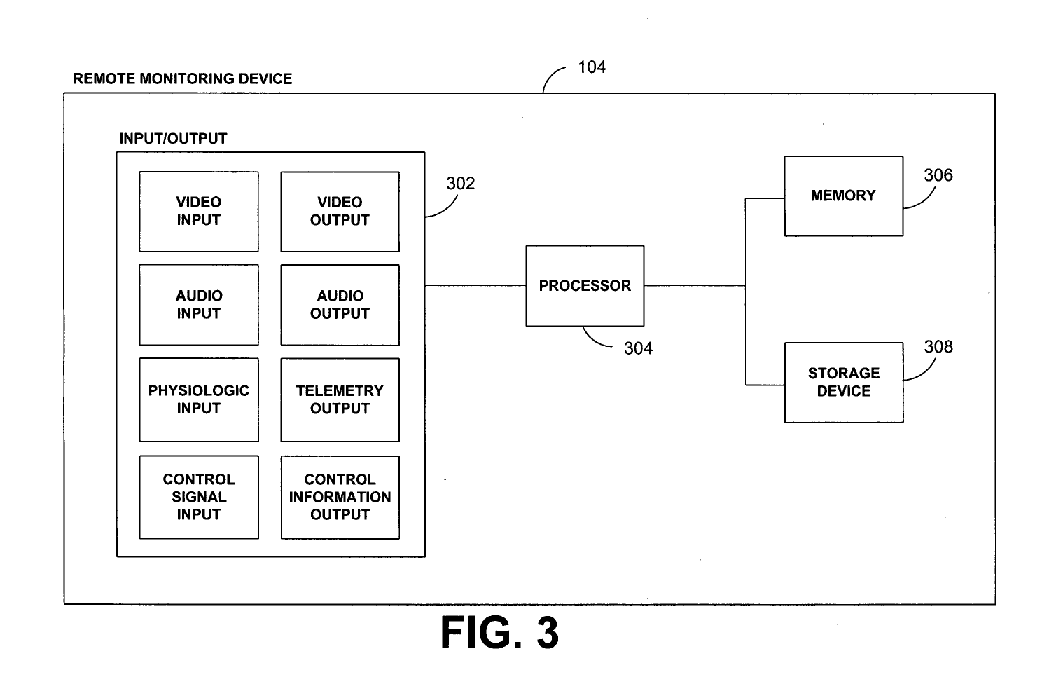

FIG. 3 is a block diagram of a remote monitoring device 104, according to an embodiment of the invention. The remote monitoring device 104 shown in detail in FIG. 3 includes components similar to those described in connection with the local monitoring component 102 (shown in FIG. 2). For instance, the remote monitoring device 104 includes I/O components 302, some of which are similar to the I/O components 202 of the local monitoring component 102. The remote monitoring device 104 also includes a processor 304, which can be the same as or similar to the processor 204 of the local monitoring component 102 (shown in FIG. 2). Similarly, a memory device 306 and storage device 308, each of which can be similar to or the same as the memory 206 and storage device 208, respectively, of the local monitoring component 102 (shown in FIG. 2).

The I/O component 302 of the remote monitoring device 104 can include various inputs, such as a video input, an audio input, a physiologic input, and a control signal input. The video and audio inputs can be used to receive video and audio information, such as from a video capture device 114 (shown in FIG. 1), an audio capture device (not shown), or other devices capable of providing video and/or audio information. The physiologic input can be used to receive physiologic information, such as vital signs or other measurable physiologic information, directly from a patient. The control signal input can be used to receive control signals output by the control signal output of the local monitoring component 102 (shown in FIG. 2).

The I/O component 302 of the remote monitoring device 104 can also include various outputs, such as a video output, an audio output, a telemetry output, and a control information output. The video output and audio output can transmit video information or data and audio information or data, respectively, which can be received by the video input and audio input, respectively, of the I/O component 202 of the local monitoring component 102. The telemetry output can be used to output data associated with a patient associated with the remote monitoring device 104 at a patient location 106, and can be received via a telemetry input of the I/O component 202 of the local monitoring component 102. Similarly, the control information output by way of the control information output of the I/O component 302 of the remote monitoring device 104 can be received by the control information input of the I/O component 202 of the local monitoring component 102 (shown in FIG. 2). The information output by way of the control information output of the I/O component 302 can be used by devices, such as the local monitoring component 102 (shown in FIG. 2) to control the remote monitoring device 104, and to assist in specifying data to be acquired by the remote monitoring device 104.

As mentioned above in connection with FIG. 1, the various outputs of the I/O component 302 of the remote monitoring device 104 can be configured to communicate via a network 108, or via a wireless access point 110. Additionally, or alternatively, the remote monitoring device 104 can communicate directly to a processor device 112, which can be collocated with the remote monitoring device 104 at a patient location 106, or can be located remotely from the patient location 106, and connected to the network 108. Additionally, information communicated by the outputs of the I/O component 302 of the remote monitoring device 104 can be communicated to any device in communication with the network 108, such as a storage device 116, or the like.

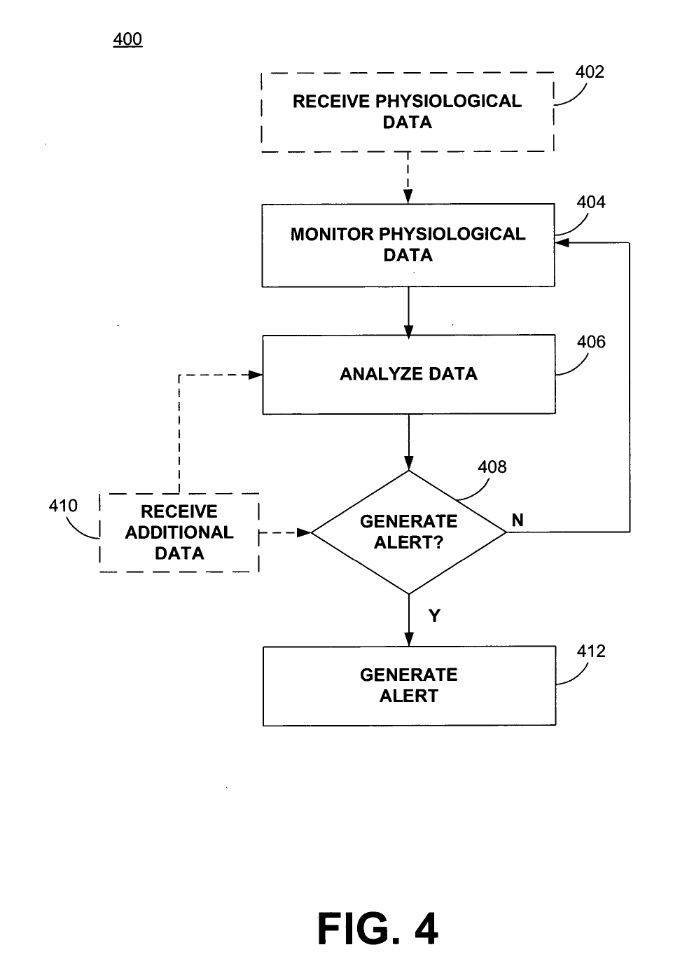

FIG. 4 is a flow diagram of an alert generation technique 400, according to an embodiment of the invention. The alert generation technique 400 shown in FIG. 4 can be performed by one or more local monitoring components 102 alone, or by one or more local components 102 in connection with one or more other components communicating with the local monitoring components 102 via the network 108. For example, all or part of the alert generation technique 400 shown in FIG. 4 can be performed by devices other than the local monitoring component 102, if desired, and the resulting alert generated by that technique 400 can be passed to the local monitoring component 102. Alternatively, the local monitoring component 102 can perform the entire alert generation technique 400 shown in FIG. 4, using processors local to the local monitoring component 102.

The alert generation technique 400 can begin by receiving physiological data in optional step 402. The physiological data (e.g., vital signs, etc.) are monitored in step 404, and that data, along with other data, are analyzed in step 406. A determination is made in step 408, regarding whether an alert should be generated. This determination can optionally be made using additional data (e.g., historical data, situational data, predictive data, etc.) that can be received in optional step 410. If it is determined that no alert is to be generated, the technique 400 continues to monitor physiological data in step 404. On the other hand, if it is determined in step 408 that an alert is to be generated, then, in step 412, an alert is generated.

One example of vital sign information that can be received in optional step 402 is a heart rate can be received in optional step 402. This information can be collected by a remote monitoring device 104 at each of the patient locations 106, and can be transmitted to a local monitoring component 102, or multiple local monitoring components 102 by way of a network 108 or a wireless network access point 110. The local monitoring component 102 can monitor the received physiological data in step 404, and can analyze the data in 406. Thus, in the simple example of a heart rate received from each patient at each patient location 106, the local monitoring component 102 can monitor the heart rates of each patient, which are continuously transmitted by the remote monitoring device 104 associated with each patient.

Upon receiving (in step 402) and monitoring (in step 404) the physiological data, the local monitoring component 102 can analyze the data in step 406, and make a determination in step 408 regarding whether or not an alert should be generated. This determination can be a simple determination based on whether a heart rate is within a predetermined desirable or acceptable range. If the heart rate is within an acceptable range, then it can be determined in step 408 that an alert need not be generated, after which the local monitoring component 102 can continue to monitor physiological data in step 404.

Additional data can be received in optional step 410, and used in the determination of step 408. For example, information associated with the specific procedures that each patient is undergoing can be received in optional step 410, and can be used in the determining of step 408. Thus, information associated with a procedure being performed on a patient, for example, can be used to determine or to adjust an acceptable predetermined range for a heart rate, which might otherwise be different. This new or adjusted predetermined range can be used in the determination of step 408 to determine whether an alert should be generated. For example, if a patient is undergoing a procedure that is known to increase heart rates generally, that information is taken into account, and a heart rate that might otherwise be determined in step 408 to require an alert to be generated, may not generate an alert under those circumstances. Additionally, the determination made in step 408 can be aided by other types of information, such as patient-specific information (e.g., a normally higher heart rate, a good tolerance for a higher heart rate, etc.), other procedure-specific information (e.g., affects of drugs being used in a procedure on the patient's heart rate), and other types of information.

The example of a heart rate given above is only one example of the type of physiological data or other patient parameters that can be monitored and analyzed and used to determine whether an alert should be generated according to the alert generation technique 400 of FIG. 4. For example, all vital signs (many of which are mentioned above) and other information that can be collected by the remote monitoring device 104 can be used in determining whether an alert should be generated.

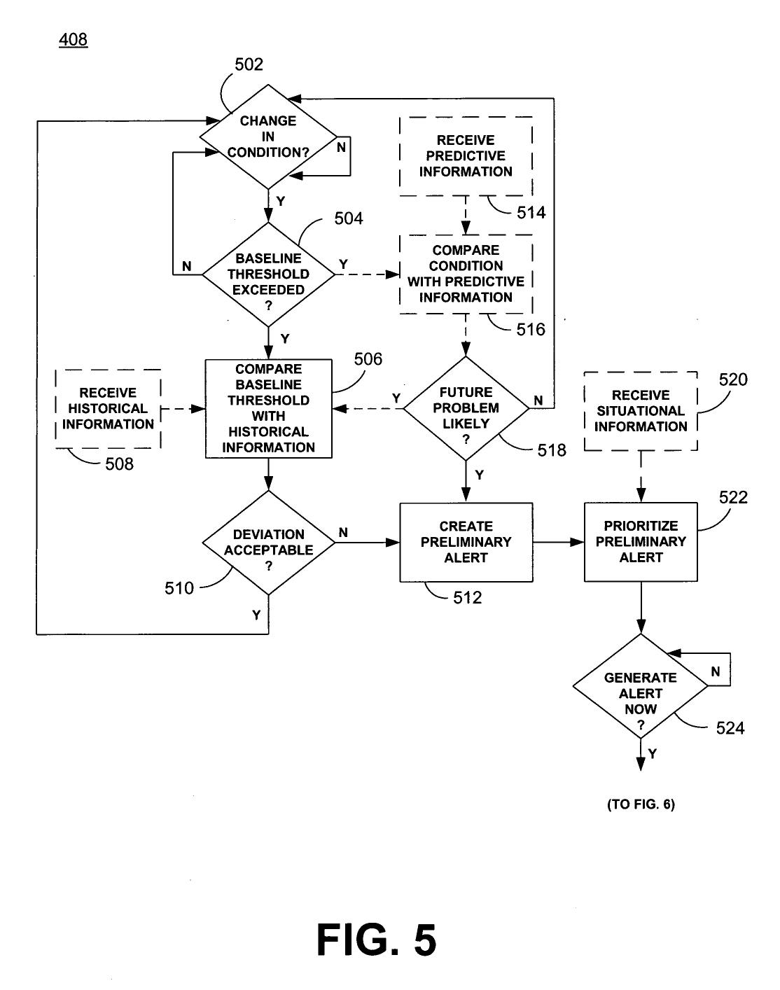

FIG. 5 is a flow diagram of an alert determination technique 408, according to an embodiment of the invention. The alert determination technique 408 of FIG. 5 begins with a determination 502 of whether a condition of a patient has changed, based on physiological data monitored in step 404 and analyzed in step 406 of the alert generation technique 400 of FIG. 4. If it is determined in step 502 that no change in a patient's condition has occurred, then the condition of the patient is continually monitored (repeating the determination of step 502), until a change in condition has occurred.

Once it is determined in step 502 that a change in condition has occurred for a patient, another determination is made in step 504 regarding whether a baseline threshold has been exceeded. If it is determined in step 504 that the baseline threshold has not been exceeded, then the determination in step 502 is repeated until another change in condition occurs. Returning to the example of a patient with an elevated heart rate, the heart rate of the patient is monitored until it is determined to have changed in step 502. If the new heart rate does not exceed a baseline threshold as determined in step 504 (i.e., the changed heart rate is still within acceptable parameters), the determination in step 502 is repeated until the heart rate changes again.

Once it has been determined in step 504 that the baseline threshold has been exceeded, then the baseline threshold is compared with historical information in step 506. The historical information can be received in optional step 508 prior to the determination of step 506. After the comparison performed in step 506, a determination is made in step 510 regarding whether the deviation of the patient's changed condition from the baseline threshold is acceptable. If the deviation is acceptable, as determined in step 510, then the patient's condition is monitored again in step 502 until it is determined to have changed.

Thus, returning to the heart rate example, if a patient's heart rate is determined in step 502 to have changed, and is determined in step 504 to be outside of a predetermined acceptable range (i.e., a baseline threshold is exceeded), that baseline threshold is compared with historical information in step 506. The historical information can be received in optional step 508, and can include, for example, patient-specific information, which may indicate, for example, that the particular patient whose heart rate is being measured is usually higher than normal, in which case it may be determined in step 510 that the deviation of the patient's changed heart rate from the baseline threshold is acceptable. Additionally, or alternatively, the historical information received in optional step 508 can also include non-patient-specific historical information, such as procedure-specific information, which can indicate, for example, that a particular procedure, or particular drugs used during a procedure, may increase a heart rate above the standard baseline threshold. If this is the case, for example, then the comparison in step 506 may yield information that causes the determination to be made in step 510 that the deviation of the patient's changed heart rate from the standard baseline threshold is acceptable because of historical information received in optional step 508.

In addition to using historical information that can be received in optional step 508, the alert determination technique 408 of FIG. 5 can also use predictive information to help determine whether an alert should be generated. For example, predictive information can be received in optional step 514. That information can be used in optional step 516 to compare a current condition of a patient with predictive information in step 516. A determination can be made in step 518, based on predictive information, whether a future problem is likely, or in other words, what the likelihood of a future problem (e.g., clinical condition, etc.) is given the current condition of a patient. If it is determined in step 518 that a future problem is likely, a preliminary alert can be created in step 512.

Predictive information can be used to compare a condition of a patient if it is determined in step 504 that a baseline threshold has been exceeded. Additionally, or alternatively, the comparison of step 516 using predictive information can be made independent of any other determinations (e.g., without requiring that a baseline threshold be determined in step 504 to have been exceeded). If it is determined in step 518 that a future problem is likely based on predictive information, the comparison in step 506 with historical information can be triggered, and/or a preliminary alert can be generated.

For example, if a condition determined to have changed in step 502 is a heart rate, and that change is an increase in the heart rate above the baseline threshold, as determined in step 504 (i.e., the heart rate is above a standard, acceptable range), then in addition to the comparison with historical information in step 506, a comparison with predictive information can also be made in step 516 (e.g., using statistical correlation or other suitable fitting techniques). Either comparison 506, 516 can independently trigger the creation of a preliminary alert 512, which may ultimately cause the generation of an alert.

For example, if the heart rate of a patient is determined to be above the baseline threshold in step 504, and the determination in step 510 indicates that the deviation between the patient's increased heart rate and the baseline threshold is acceptable based on historical information, an alert still can be generated based on predictive information. This may be the case, for example, if predictive information received in optional step 514 indicates that an increased heart rate combined with an increased blood pressure exist can indicate a high likelihood of the development of cardiac ischemia or intraoperative awareness. Based on this predictive information, the patient's condition would be compared with the predictive information in step 516 (i.e., the patient's heart-rate and blood pressure would be compared with the predictive information). If, after this comparison, it is determined in step 518 that a future problem, such as cardiac ischemia or intraoperative awareness, is likely (i.e., that the likelihood of a future problem exceeds a predetermined threshold probability), then a preliminary alert would be generated in step 512.

There are numerous examples of predictive information that can be used according to one or more embodiments of the invention to determine the probability of a condition occurring in the future. According to one or more embodiments of the invention, this predictive information can be adaptively learned based on historical information (including patient-specific information, non-patient-specific, procedure specific information, non-procedure-specific information, etc.), such that when potential indicators and developed conditions are observed in multiple patients, predictive information of the system can be updated. For example, if it is determined by observation, or if information is entered by a healthcare provider indicating that decreasing oxygen saturation intraoperatively is likely to lead to respiratory insufficiency postoperatively or possible respiratory arrest or intubation postoperatively, predictive information can be updated and alerts can be generated based on that predictive information. As future observations confirm or refute the correlation, the predictive information can be modified either by the system or by a user (e.g., a clinician). As another example, increasing airway pressure intraoperatively may indicate a likelihood of bronchospasm or wheezing, which can be used to create predictive information upon which an alert can be based, according to one or more embodiments of the invention. Additionally, because low anesthetic levels (as measured by concentrations of inhaled anesthetic agents, administered anesthetics, such as narcotics), can lead to postoperative recall of intraoperative events, when low anesthetic levels are observed, an alert can be generated based on the predictive information and likelihood of a future problem. Other examples of predictive information that can be entered by a clinician exist, and predictive information not currently known may also be added as correlations are observed.

In addition to using historical information and predictive information, situational information can also be used to assist in the determination of whether an alert should be generated. For example, once it is determined that in step 510 that a deviation of a patient's changed condition from a baseline threshold is not acceptable in view of historical information, and/or it is determined in step 518 that a future problem is likely based on predictive information, a preliminary alert is created in step 512. This preliminary alert might be one of many preliminary alerts or actual alerts being handled by the system. Thus, the preliminary alert created in step 512 may need to be handled in view of situational information received in optional step 520.

In step 522, each preliminary alert can be prioritized based on the situational information received in optional step 520. Thus, for example, if the preliminary alert generated in step 512 is the least urgent of a group of preliminary alerts being generated according to the alert determination technique 408, then in step 522, it may be assigned the lowest priority, and may be handled last among those alerts. This may mean that an actual alert corresponding to that preliminary alert is not be generated until more urgent alerts have been generated and handled by healthcare providers, or it may mean that it generates an alert (in step 524) that has a lower priority than other alerts similarly generated.

The situational information received in optional step 520 and used to prioritize preliminary alerts in step 522 can include a variety of situational information. For example, priorities of the preliminary alerts, conditions for which patients are being treated, procedures by which patients are being treated, and/or priority levels of available healthcare providers can be taken into account in prioritization of preliminary alerts in step 522. Additionally, other situational information, such as scheduling information, which can be received from third-party software packages, for example, can be taken into account. For example, if a relatively non-urgent, low-level preliminary alert is created in step 512, and one or more critical procedures requiring immediate attention is about to begin, an alert may not be generated in step 524 until after the procedures requiring immediate attention have been addressed. Thus, the preliminary alert would be prioritized in step 522 below the immediate priority of handling the urgent procedure.

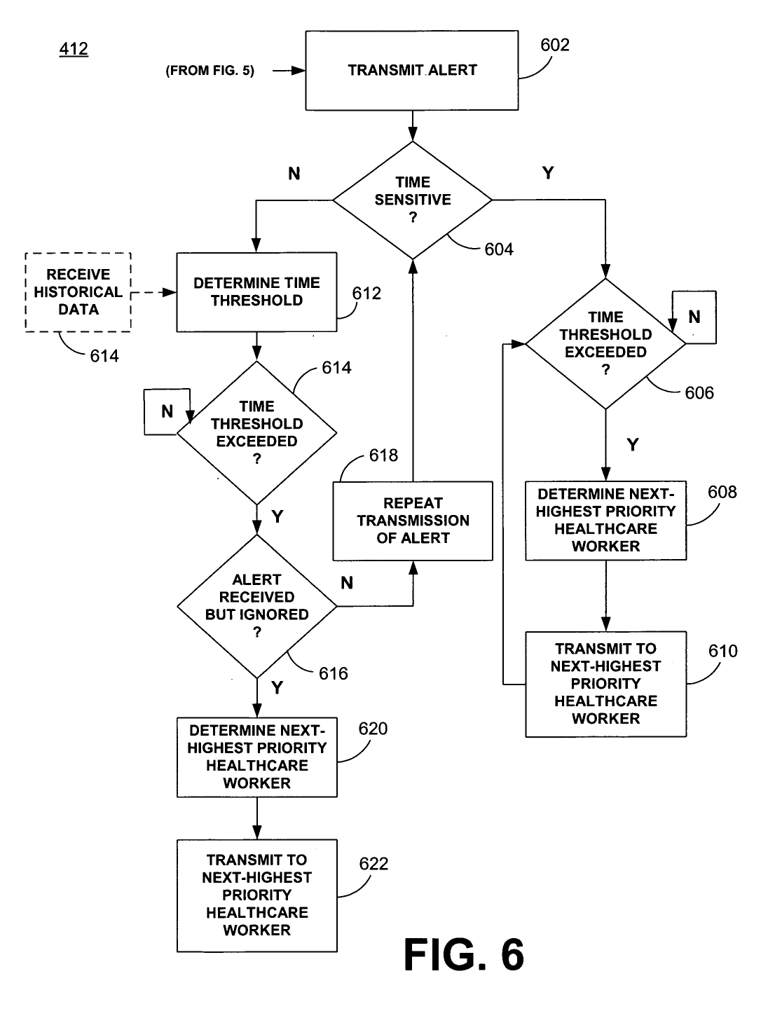

Once a preliminary alert is prioritized in step 522, a determination is made in step 524 regarding whether or not the alert should be generated now. If it is determined that the alert should not be generated now, then the determination in step 524 is repeated until it is time to generate the alert. Once it is determined in step 524 that it is time to generate the alert, then the process continues in the alert generation technique 412 of FIG. 6.

FIG. 6 is a flow diagram of an alert generation technique 412, according to an embodiment of the invention. The alert generation technique 412 of FIG. 6 begins as an alert is transmitted in step 602, after the determination is made in step 524 (shown in FIG. 5) that an alert should be generated. As discussed above, transmission of an alert can be accomplished by way of a network 108, a wireless network access point 110, or other suitable technique. Depending upon the desired functionality of the system within which the alert is being generated, the alert generated in step 602 can also, optionally, be addressed to a particular local monitoring component 102, or to a practitioner using a local monitoring component 102.

Once the alert has been transmitted in step 602, a determination is made in step 604 regarding whether or not the alert transmitted in step 602 is time sensitive. If it is determined that the alert is time sensitive, then an additional determination is made in step 606 regarding whether a predetermined time threshold for responding to the alert has been exceeded without the alert being addressed. If the time threshold has not been exceeded, as determined in step 606, the system continues to check until the time threshold is exceeded. Once the time threshold has been exceeded without the alert being addressed, as determined in step 606, the next-highest priority healthcare worker is determined in step 608, and the alert is transmitted to that next-highest priority healthcare worker. In other words, if an alert is transmitted to a particular local monitoring component 102, or addressed to a healthcare practitioner, and the practitioner using the local monitoring component 102 does not respond to the alert within a predetermined time threshold, then the alert is escalated to the next available healthcare provider. The determination of which healthcare provider is next to be notified can be made according to a predetermined priority list of healthcare providers, for example.

Once the alert has been escalated to the next-highest priority healthcare worker in step 610, the determination in step 606 is repeated until the alert is responded to, or until the time threshold has been exceeded again. Once the time threshold has been exceeded again, then the next-highest priority healthcare worker is determined in step 608 and the alert is transmitted to that next-highest priority healthcare worker in step 610. In this manner, an alert that does not receive a response within a predetermined time threshold continues to be escalated and broadcast to additional healthcare providers until the alert is responded to within the time threshold, as determined in step 606. If the alert is escalated to all available healthcare workers, and has not been addressed, or no response has been received, then the alert can be repeated and re-sent to the healthcare workers, beginning again with the healthcare worker who first received it.

If it is determined in step 604 that the alert transmitted in step 602 is not time sensitive, then a time threshold for response is determined in step 612. This time threshold determined in step 612 is the time within which the alert generation technique 412 requires a response to the alert transmitted in step 602 prior to taking additional action. The time threshold determined in step 612 can be based upon additional data, such as historical data, received in optional step 614.

Returning to the example of a patient with an elevated heart rate, if a slightly elevated heart rate causes the alert to be transmitted in step 602 (i.e., the alert determination technique 408 of FIG. 5 determines that an alert should be generated), but it is determined in step 604 that the slight elevation in heart rate is not time sensitive condition, then a time threshold is determined in step 612. This time threshold can be based on historical data, such as patient-specific data, which can be optionally received in step 614. For example, a longer time threshold may be determined in step 612 if the historical data received in optional step 614 indicate that the patient for whom the alert has been generated has a higher than normal heart rate and, therefore, the slightly elevated heart rate that caused the alert to be generated is not a time sensitive or urgent condition, and the patient may be able to comfortably and safely endure the slightly elevated heart rate in view of the historical data. On the other hand, a different set of historical data might cause the time threshold to be shorter.

Once a time threshold has been determined in step 612, a determination is made in step 614 regarding whether the time threshold has been exceeded. If the time threshold has not been exceeded, then the alert generation technique 412 waits until the time threshold has been exceeded, as determined in step 614. If the alert has not been responded to within the time threshold, and thus the time threshold is determined in step 614 to have been exceeded, then an additional determination is made in step 616 regarding whether the alert was received but ignored (e.g., if the alert was viewed but dismissed without further action by a healthcare provider). If it is determined that the alert was received but not necessarily ignored in step 616, then transmission of the alert can be repeated in step 618, and the determination can be made in step 604 again regarding whether or not the alert is time sensitive.

Thus, according to the alert generation technique 412 of FIG. 6, if a healthcare provider has received the alert transmitted in step 602 and that alert is not time sensitive, even though the healthcare provider has not yet addressed the alert, as long as the alert has not been dismissed or otherwise ignored, that healthcare provider may still be allowed to address the concern raised by the alert (unless has become time sensitive, as determined when step 604 is repeated). In other words, because the alert is not time sensitive, there is no need to escalate it to the next healthcare provider. However, once it is either determined (in step 616) that the alert has been received but ignored and the time threshold has been exceeded (as determined in step 614), or that the alert has become time sensitive (as determined in step 604) and the time-sensitive threshold has been exceeded, then the next-highest priority healthcare worker is determined in step 620 and the alert is transmitted to that next-highest priority healthcare worker in step 622 (or, if the alert has become time sensitive, in steps 608 and 610).

Continuing the example of a patient with a slightly elevated heart rate, if it is determined in step 614 that the time threshold has been exceeded, despite the fact that the alert may not be time sensitive, then an additional determination is made in step 616 regarding whether the alert has been received but ignored. If the alert has been received and ignored, as determined in step 616, then the alert is escalated to the next healthcare provider in step 622 (who is determined in step 620). If the alert has been received but apparently not ignored, as determined in step 618, indicating that the healthcare provider receiving the initial transmission of the alert may still intend to respond to the alert, then no escalation is made, unless it is determined in step 604 that the alert has become time sensitive, or until the alert is received but ignored, as determined in step 616.

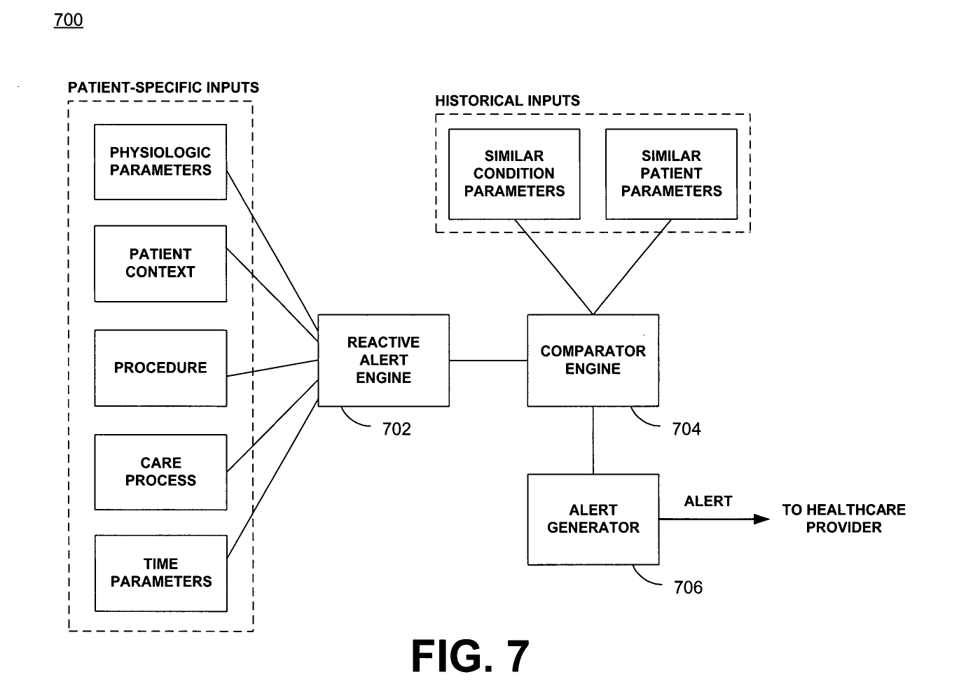

FIG. 7 is a block diagram of a data analysis system 700, according to an embodiment of the invention. The data analysis system 700 includes three main components: a reactive alert engine 702, a comparator engine 704, and an alert generator 706. The reactive alert engine 702 receives various patient-specific inputs. For example, the reactive alert engine 702 can receive physiologic parameters associated with a particular patient, such as vital signs, or the like. Additionally, the reactive alert engine 702 can receive patient context information, including such information as medical history and profile information, information about specific risk factors, and other patient-specific background information, which can be used to determine whether or not an alert (or preliminary alert) should be generated by the reactive alert engine 702. Additionally, the reactive alert engine 702 can take into account information about a specific procedure that a patient is undergoing, including information about how the procedure generally interacts with patients' vital signs or other patient parameters, for example.

The reactive alert engine 702 can also receive and use information associated with a care process for a patient and time parameters associated with a patient. For example, care process information can include information about where a patient is in the perioperative process (e.g., including pre-operative, operative, and post-operative procedures and processing). For example, care process alerts can include alerts such as “patient arrived in holding room,” “surgical incision made,” or other similar alerts. This care process information can be entered, for example, by healthcare providers at a patient location (e.g., a nurse, intern, physician, etc.). Time parameters used by the reactive alert engine 702 can include time calculations for a patient based on care process information, such as a calculation of actual or predicted times or lengths of events in the care process. For example, the time parameters can include such parameters as length of time in an OR, time until or time since surgical incision, or other time parameters.

The reactive alert engine 702 can generate alerts, for example, based on physiologic parameters, using other patient-specific information, such as patient context information, procedure information, care process information, and/or time parameters information as filters for understanding the physiologic parameters. Returning to the example of a patient with the slightly elevated heart rate, the reactive alert engine 702 might not generate an alert for such a patient if the patient's slightly elevated heart rate might be considered normal in view of additional patient-specific inputs received, including other physiologic parameters being monitored, a patient context (e.g., a history of slightly elevated heart rate), procedure information, care process information, or time parameters information.

Once the reactive alert engine 702 has determined than an alert should be generated, that information is passed to the comparator engine 704. The comparator engine 704 receives additional historical inputs, and uses those inputs to determine whether or not to pass the information received from the reactive alert engine 702 to the alert generator 706. For example, the comparator engine 704 can use historical inputs, such as similar condition parameters or similar patient parameters, to determine whether to pass information from the reactive alert engine 702 to the alert generator 706. For instance, parameters associated with a condition similar to a condition of a patient for which the reactive alert engine 702 indicates that an alert should be generated can be used by the comparator engine 704 to determine whether an alert should be generated. Likewise, the comparator engine 704 can use information from similar patients or example patients (including hypothetical patients) to determine whether the alert indicated by the reactive alert engine 702 should be generated. If the comparator engine 704 determines, based on historical inputs, that an alert should be generated, then the alert generator 706 is notified, and an alert is generated to the healthcare provider (e.g., by way of a local monitoring component 102).

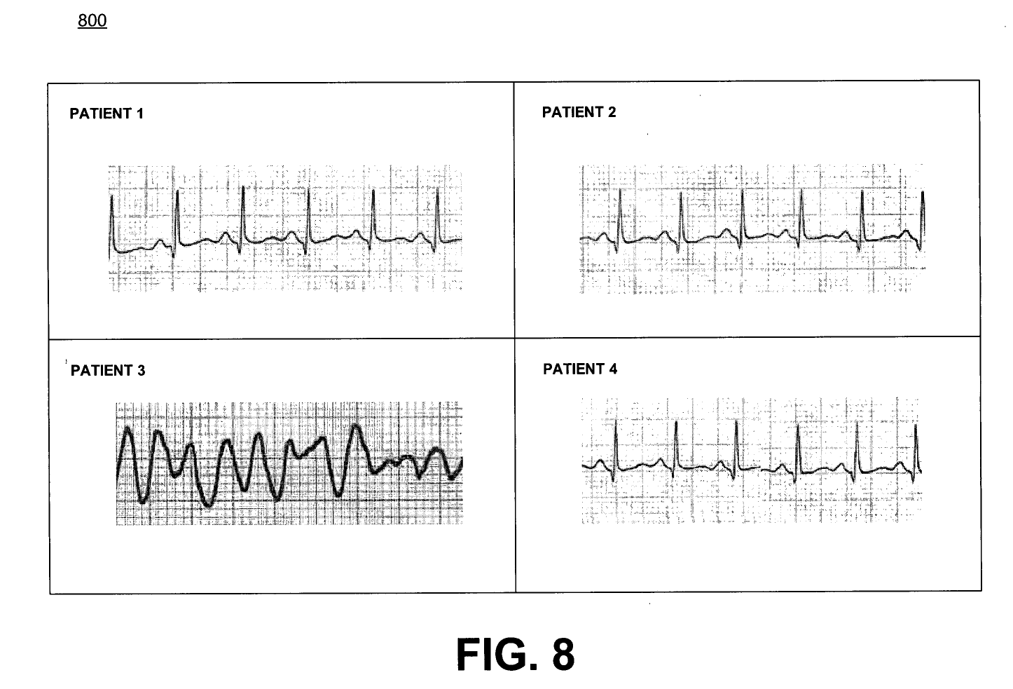

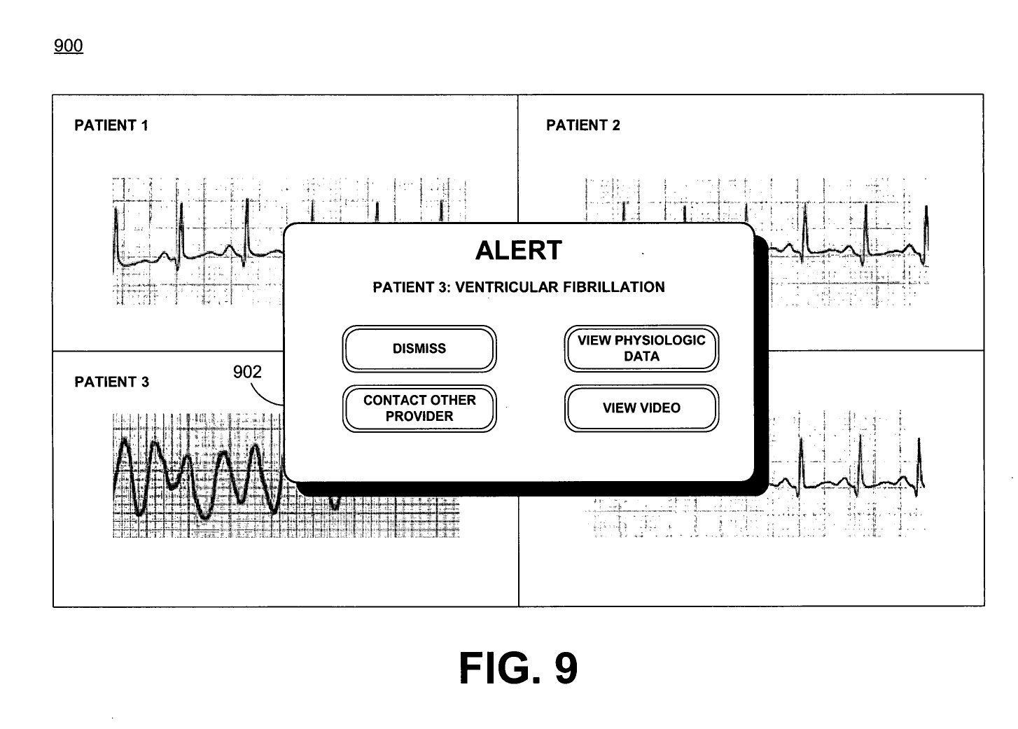

FIG. 8 is a block diagram of a display 800 showing vital signs of multiple patients, according to an embodiment of the invention. The display 800 illustrated in FIG. 8 shows a multi-patient view provided by a local monitoring component 102 (shown in FIG. 1), wherein ECG values are shown for four patients: Patient 1, Patient 2, Patient 3, and Patient 4. As can be clearly seen in FIG. 8, the heartbeat of Patient 3 is irregular. Thus, simply by being able to monitor the heart beats of multiple patients simultaneously using the display 800 shown in FIG. 8, a healthcare provider is able to more quickly identify the irregular heart beat of Patient 3. Additionally, as described above, one or more embodiments of the invention provide a technique whereby an alert is generated when conditions warrant that an alert be generated. Thus, even if a healthcare provider viewing the display 800 shown in FIG. 8 does not recognize the irregular heart beat of Patient 3, an alert, similar to the alert shown in FIG. 9 will be generated.

FIG. 9 is a block diagram of a display showing an alert generated by an alert generation system, according to an embodiment of the invention. The display 900 shown in FIG. 9 is the same display 800 shown in FIG. 8, with the added alert shown in the pop-up window 902, which overlays the display 800 shown in FIG. 8. By providing a pop-up window 902 to alert a healthcare provider of the irregular heart beat of Patient 3, the healthcare provider will be required to address the alert, prior to fully viewing the data associated with the various patients. The alert shown in the pop-up window 902 in FIG. 9 is only one example of alerts that can be generated, and various other information can be provided by way of such alerts. According to one or more embodiments of the invention, alerts can be customized by one or more healthcare providers to suit the needs of a particular facility, patient, patient population, healthcare provider, group of healthcare providers, or other entities, as desired.

It should be noted that the pop-up window 902 of the alert can be allowed to be moved or resized, according to various constraints of the system. Thus, a healthcare provider may be allowed to continue to monitor the irregular heartbeat of Patient 3 prior to dismissing the alert, by moving the window 902 of the alert to a location where it does not obscure the information being displayed for Patient 3. Additionally, the window 902 of the alert can be made either modal or non-modal, depending upon the seriousness of the alert, or other factors. For example, if it is determined in step 604 of FIG. 6 that the alert in the window 902 is time sensitive (as it likely would be in the situation illustrated in FIG. 9), then the alert window 902 can be made modal, such that no further action can be taken (with the possible exception of moving or resizing the alert window 902) until the alert is addressed. On the other hand, if it is determined that the alert within the window 902 is not time sensitive (e.g., in step 604 of FIG. 6), then the alert window 902 can be made non-modal, such that a healthcare provider can continue to interact with the underlying application, prior to addressing the alert in the alert window 902.

As shown in FIG. 9, there are several options for responding to the alert. The options shown in FIG. 9 are merely examples, and additional actions can be included if desired. The window 902 shows four example buttons, indicating four different example actions for responding to the alert. First, a user (e.g., a healthcare professional) can dismiss the alert by pressing the “dismiss” button. A user may wish to press the dismiss button if the user is attending to something more urgent than the alert generated. When the dismiss button is selected, the alert will be removed (i.e., the display will again appear as the display 800 shown in FIG. 8) and will be interpreted as received but ignored (e.g., in step 616 of FIG. 6).

A user can also select the button “contact other provider” if it is desirable that the alert be transmitted to another healthcare provider. This may be the case, for example, if the user is attending to something urgent that cannot be postponed to address the problem indicated in the alert. Additionally, the user may wish to obtain additional information about the patient for whom the alert is generated. In such cases, the healthcare provider can press the “view physiologic data” button, which will cause the data that is causing the alert to be generated to be displayed to the user. This is particularly useful, in cases where an alert is generated for data that is not readily viewable (e.g., because it is covered by the alert window 902). Similarly, a user may wish to view video of the patient for whom the alert is generated, and can do so by selecting the “view video” button, which will cause real-time video of the patient for whom the alert is generated to be shown. This can be useful, for example, in viewing the treatment the patient is receiving to address the problem that has generated the alert, to view which practitioners are assisting the patient, or to learn other information that can be obtained by way of such real-time video.

As shown in FIG. 9, Patient 3 is experiencing ventricular fibrillation, which is a serious, life-threatening condition. To generate the alert shown in FIG. 9, the alert generation technique 400 shown in FIG. 4 is followed, as physiological data is received in step 402 (e.g., the ECG for each patient) and monitored in step 404. That data is analyzed in step 406, and a determination is made regarding whether an alert should be generated in step 408. Because the data associated with Patient 3, when analyzed in step 406 of FIG. 4, is so serious, there may be no need to receive additional data prior to determining whether an alert should be generated, and the alert is immediately generated in step 412, after determination that the patient is undergoing a ventricular fibrillation.

The alert determination technique 408 shown in FIG. 5 proceeds rapidly, as it is determined in step 502 that a changing condition has occurred, and in step 504 that the baseline threshold has been exceeded. To the extent that historical information can be used to identify the condition of Patient 3 rapidly, that information may be received in step 508 and used for comparison in step 506. In step 510 of FIG. 5, the deviation of the ECG pattern of Patient 3 from the baseline threshold would not be considered acceptable, and a preliminary alert would be generated in step 512. Based on predictive information as well, received in optional step 514, a future problem would be determined to be likely in step 518 based on the predictive information, and would independently cause a preliminary alert to be created in step 512. The preliminary alert would be prioritized in step 522 according to situational information, and because of the seriousness of the condition would likely be the highest priority preliminary alert, such that an alert would be generated immediately in step 524.

The technique would continue in the alert generation technique 412 shown in FIG. 6, whereby an alert would be transmitted, in step 602 and treated as time sensitive, as determined in step 604. The time threshold used in the determination of step 606 would be extremely short for such a serious condition, and the alert would be escalated to other healthcare providers unless the alert is responded to within the short time threshold, as determined in step 606.

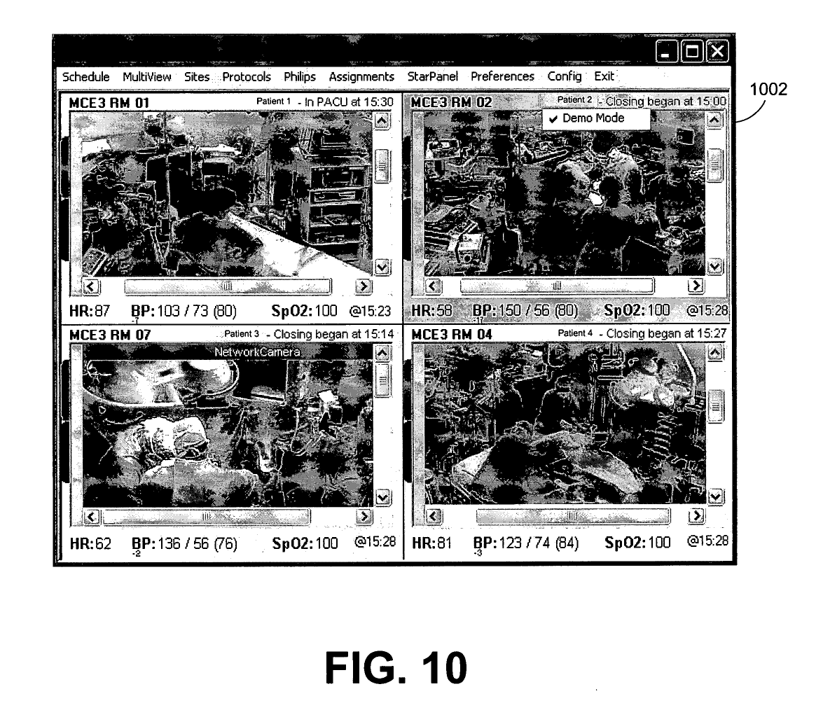

FIG. 10 is a screen shot of a display showing video of multiple patients (Patient 1, Patient 2, Patient 3, and Patient 4) during procedures, according to an embodiment of the invention. The window 1002 of FIG. 10 shows real-time video of the multiple patients undergoing various procedures. This real-time video of multiple patients can be viewed using the local monitoring component 102, according to one or more embodiments of the invention. The local monitoring component 102 can transmit a control signal output to control the cameras or other video capture devices 114 in the various patient locations 106 (shown in FIG. 1) to control video size, pan, tilt, zoom, or other controls of the video capture device 114. Thus, the healthcare provider using the local monitoring component 102 can remotely control the remote monitoring device 104 and/or the video capture device 114 by way of a control signal output of the IO component 202 of the local monitoring component 102. In this manner, a healthcare provider can control the view of the video capture device, and see things that are needed to be seen for proper diagnosis and/or treatment of the patients for whom the video is being viewed.

As shown in the window 1002 of FIG. 10, each video pane is identified by a patient identifier and an operating room number for quick reference. Additionally, status information regarding the procedure being performed is shown in the upper right hand corner, and is entered by a healthcare provider at the patient location 106. Vital signs and other parameters for the patient are also displayed, including heart rate, blood pressure, and pulse oximetry values. The vital signs shown in the window 1002 of FIG. 10 are merely examples, and additional, alternative vital sign values can be displayed as well.

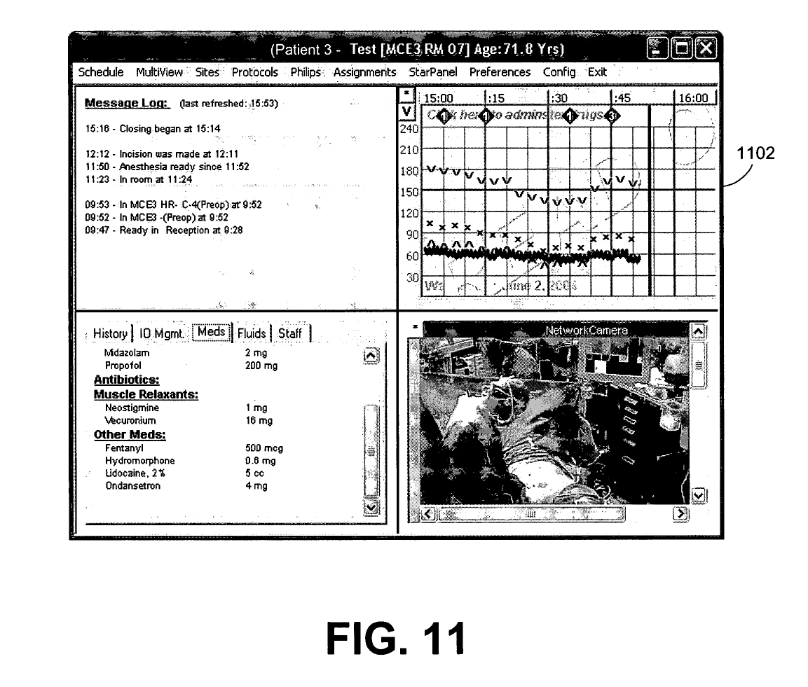

FIG. 11 is a screen shot of a display showing various information of a single patient, according to an embodiment of the invention. The window 1102 shown in FIG. 11 illustrates a different, patient-specific view (for Patient 3) that can be obtained using the local monitoring component 102. The view shown in the window 1102 of FIG. 11 includes a message log regarding the procedure being performed in the upper-left-hand quadrant. Information specific to the patient (Patient 3) being monitored, including medications, history, and other useful information is shown in the bottom-left-hand quadrant. Various measured parameters are shown graphically in the upper-right-hand quadrant, such as systolic and diastolic blood pressure, mean or average blood pressure, heart rate, drug administration times or rates, or other parameters of interest. In the lower-right-hand quadrant, real-time video of the procedure involving Patient 3 is displayed. As with the window 1002 of FIG. 10, the video displayed in the lower right hand quadrant of the window 1102 of FIG. 11 can be controlled by the local monitoring component 102, which can cause the video capture device to pan, tilt, zoom, or otherwise change the video being captured.

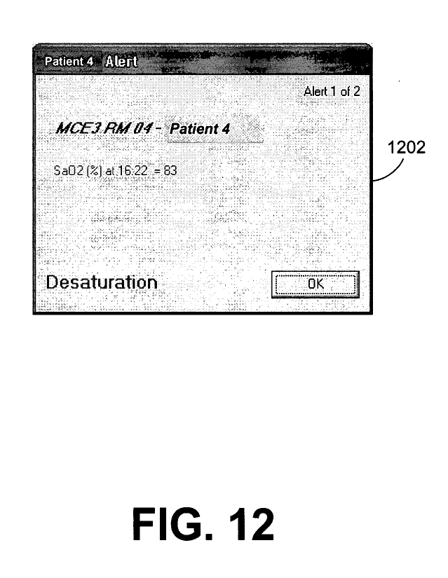

FIG. 12 is a screen shot of an alert generated by an alert generation system, according to an embodiment of the invention. The window 1202 shown in FIG. 12 is an example of a pop-up window used to communicate an alert generated for one of the patients (Patient 4) for whom video was displayed in FIG. 10 and an ECG signal was displayed in FIG. 11. The alert in the window 1202 shown in FIG. 12 indicates that the oxygen saturation for that patient has decreased, and may require immediate attention. Additionally, the alert shows the OR location of the patient (Patient 4).

A user of the local monitoring component 102 by which the alert window 1202 shown in FIG. 12 is displayed can cycle through various views according to one or more embodiments of the invention. For example, a user could first view the multi-patient video view shown in FIG. 10, followed by the window 1102 for a single patient (Patient 3) shown in FIG. 11, where the patient for whom the alert is eventually generated (e.g., Patient 4) is not visible. That is, while viewing the window 1102 of FIG. 11 showing information about Patient 3, a user of the local monitoring component 102 is not aware of the changing vital signs of Patient 4, for whom the alert shown in FIG. 12 is ultimately generated. However, because of the automatic monitoring and alert generation of various embodiments of the invention, the alert for Patient 4 shown in FIG. 12 is generated in a manner such that a healthcare provider can act upon that alert in a timely fashion regardless of whether or not the healthcare provider was aware of the condition of that caused the alert to be generated. Thus, the constant monitoring and alert generation capability of various embodiments of the invention allow a healthcare provider to rely on the alert generation system to be vigilant in alerting the healthcare provider of potential problems. Because of this vigilance capability of the system, healthcare providers, such as anesthesiologists or other clinicians, can treat multiple patients, increasing their efficiency, with increased confidence, and a decreased risk of error.

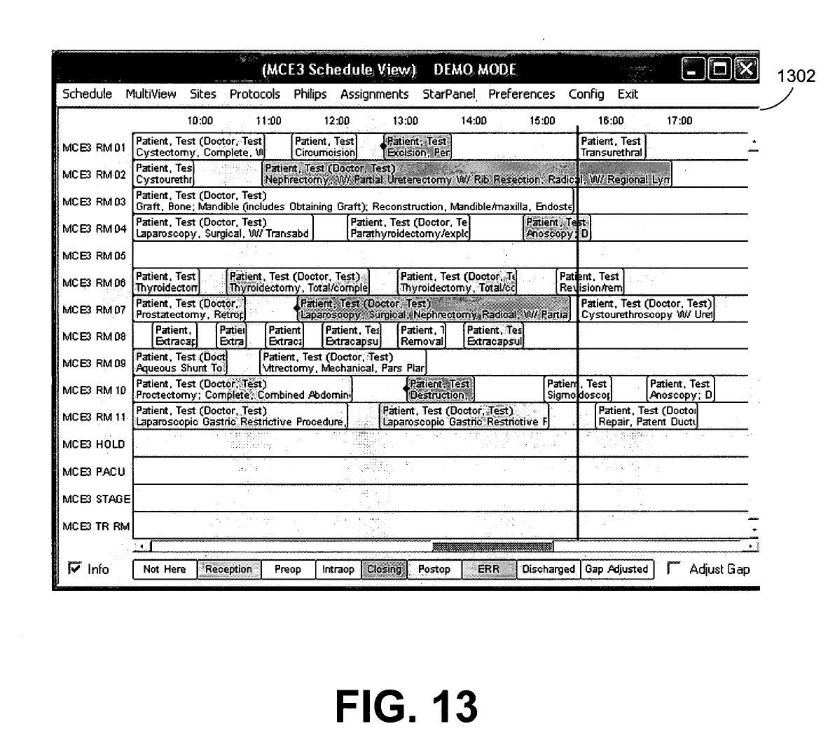

FIG. 13 is a screen shot of a multi-patient healthcare schedule, according to an embodiment of the invention. The window 1302 shown in FIG. 13 illustrates scheduling for multiple operating rooms. As can be seen in the window 1302 shown in FIG. 13, various procedures are shown, and a vertical line representing the current time is illustrated relative to all of the procedures. The scheduling information shown in FIG. 13 is helpful to healthcare providers using the local monitoring component 102 as they can readily ascertain the various procedures being performed, or scheduled to be performed, and maintain an organized approach to visiting or remotely monitoring each of those patients as necessary. The status of various patients and related procedures can be entered locally or remotely by one or more healthcare providers, or can be automatically generated as a patient is registered in different locations.

Data associated with the schedule shown in the window 1302 of FIG. 13 can be used as situational information in connection with the various techniques described above for generating alerts based on situational information. To aid healthcare providers, the various procedures illustrated in the schedule shown in the window 1302 of FIG. 3 can be color-coded such that, for example, the stage of each operation displayed is readily apparent. For example, procedures currently in the final stages of closing can be color-coded with a first color, while intraoperative procedures can be color coded a different second color. Similarly, different color codes can be used for various other stages of procedures, including preoperational, reception, post-operational, emergency room treatments, and discharged patients. Because this information is available via the local monitoring component 102 (shown in FIG. 1), which can form part of a portable computing device used by a healthcare provider, the schedule shown in the window 1302 of FIG. 13 can be with the healthcare provider at all times, and (in combination with alerts, etc.) can aid the provider in determining how best to allocate time in treating patients most efficiently and effectively.

From the foregoing, it can be seen that systems and methods for remote monitoring of multiple healthcare patients are discussed. Specific embodiments have been described above in connection with a portable computing device to be used by a healthcare practitioner to remotely monitor multiple patients simultaneously.

It will be appreciated, however, that embodiments of the invention can be in other specific forms without departing from the spirit or essential characteristics of the invention. For example, while the foregoing examples have focused principally on hospital or operation room management within a hospital, the principles of the invention can also be readily applied in other healthcare contexts. Conditions other than the conditions given as examples above (e.g., a patient with an increased heart rate) can be similarly applicable using the principles described above.

Additionally, other applications outside of the healthcare environment, for which it would be desirable to monitor multiple remote locations simultaneously, can make use of the principals described herein. For example, various embodiments of the invention can also be used within a security context, wherein a security guard or other security personnel can use a portable computing device or other local monitoring component to simultaneously monitor multiple locations (e.g., using multiple remote monitoring devices), and to receive alerts from the system when those alerts are generated (e.g., in response to a breach of security or other security events of interest). Much like the healthcare embodiments described in detail above, an embodiment of the invention used for security purposes can also make use of dynamic or adaptive determination (e.g., using information such as historical information, situational information, and predictive information) of whether alerts should be generated and to whom they should be sent or addressed.

Similarly, one or more embodiments of the invention can be used within an inventory management environment. For example, one or more inventory managers or other individuals can use a portable computing device, or other local monitoring component, to simultaneously monitor multiple inventory locations or other locations of interest to an inventory manager. Each of the locations of interest can have a remote monitoring device, a video capture device, or other components as necessary, which transmit data and information to the inventory manager. Using such a system, an inventory manager or other individual can potentially monitor, approve, and/or record the flow of inventory items. Additionally, using a remote monitoring device to acquire data about such locations, an inventory manager or other interested individual can monitor data as it relates to the inventory. For example, for inventory that requires temperature control, an inventory manager can monitor data regarding the temperature of inventory items. Alerts can be generated if temperatures exceed temperature control thresholds, and those alerts can be based on historical, situational, and/or predictive information or data, in a manner similar to the healthcare embodiments described above. Alerts regarding other conditions can also be generated in a similar manner.

It also should be recognized that, although certain functionalities and components described herein have been described as specifically pertaining to or being implemented by hardware or software, to the extent possible, hardware functionalities and components are intended to be interchangeable with software functionalities and components, and vice versa.

The presently disclosed embodiments are, therefore, considered in all respects to be illustrative and not restrictive.

What is claimed is:

1. A method, comprising:
monitoring data associated with a plurality of patients;
simultaneously displaying data associated with at least two of the plurality of patients;
determining if an alert should be generated for a patient from the plurality of patients at least partially based on data associated with the patient, the determining being further based on a comparison of a potential priority of the alert and a priority of the data being displayed, the potential priority of the alert being at least partially based on the data associated with the patient; and
generating an alert in substantially real-time for the patient if it is determined that an alert should be generated for the patient.

2. The method of claim 1, wherein the data associated with the plurality of patients includes data associated with vital signs of the plurality of patients.

3. The method of claim 1, wherein the data associated with the plurality of patients is simultaneously displayed on a single portable display.

4. The method of claim 1, wherein the determining is further based on at least one of a comparison of the potential priority of the alert and a schedule of procedures, and a comparison of the potential priority of the alert and an availability of healthcare providers.

5. The method of claim 1, wherein the determining is further based at least one of historical information, situational information, and predictive information.

6. A method, comprising:
monitoring data associated with a patient;
dynamically determining if an alert should be generated for the patient at least partially based on data associated with the patient, the dynamically determining being further based on at least one of historical information, situational information, and predictive information; and
generating an alert in substantially real-time for the patient if it is determined that an alert should be generated for the patient.

7. The method of claim 6, wherein the dynamically determining is based on medical history data for the patient.

8. The method of claim 6, wherein the dynamically determining is based on historical data for a procedure associated with the patient.

9. The method of claim 6, wherein the dynamically determining is based on scheduling data associated with a plurality of patients, the patient being from the plurality of patients.

10. The method of claim 6, wherein the dynamically determining is further based on scheduling data associated with a plurality of patients, the patient being from the plurality of patients, the generating of the alert being coordinated based on the scheduling data associated with the plurality of patients.

11. The method of claim 6, wherein the dynamically determining is further based on scheduling data associated with a plurality of patients, the patient being from the plurality of patients, the generating of the alert being coordinated based on a priority associated with each patient from the plurality of patients.

12. The method of claim 6, wherein the dynamically determining is further based on predictive information associated with the patient, the predictive information including information configured to predict the effect of a current condition of the patient on a probability of developing a future condition.

13. The method of claim 6, wherein the generated alert is addressed to a specific healthcare provider.

14. The method of claim 6, wherein the generated alert is addressed to a first healthcare provider, the alert being addressed to a second healthcare provider if the alert is not acknowledged by the first healthcare provider within a predetermined time period, the first healthcare provider and the second healthcare provider being determined according to a predetermined order of healthcare providers.

15. The method of claim 6, wherein the monitoring is configured to monitor data associated with a plurality of patients, the plurality of patients including the patient, the dynamically determining being configured to dynamically determine if an alert should be generated for a patient from the plurality of patients at least partially based on data associated with that patient, the generating being configured to generate an alert in substantially real-time for a patient from the plurality of patients if it is determined that an alert should be generated for that patient.

16. The method of claim 6, further comprising:
simultaneously displaying data associated with a plurality of patients, the plurality of patients including the patient, wherein the monitoring is configured to monitor data associated with the plurality of patients, the dynamically determining being configured to dynamically determine if an alert should be generated for a patient from the plurality of patients at least partially based on data associated with that patient, the dynamically determining also being at least partially based on a comparison of a potential priority of the alert and a priority of the data associated with the plurality of patients currently being displayed, the generating being configured to generate an alert in substantially real-time for a patient from the plurality of patients if it is determined that an alert should be generated for that patient.

17. An apparatus, comprising:
a receiver configured to receive transmissions from a plurality of monitors, the transmissions from each monitor from the plurality of monitors including data associated with a patient uniquely associated with that monitor;
a processor configured to analyze transmissions received from each monitor from the plurality of monitors, the processor being configured to dynamically determine if an alert for a patient associated with a monitor from the plurality of monitors should be generated based on at least one of historical data, situational data, and predictive data, the processor being further configured to generate an alert for the patient, if it is determined that an alert should be generated; and
a display configured to display information associated with the transmissions received by the receiver according to instructions received from the processor, the display being configured to cause selected information associated with the transmissions to be displayed, the display being further configured to display any alerts generated by the processor.

18. The apparatus of claim 17, wherein the processor is configured to determine if an alert should be generated based on at least one of historical data associated with a patient and historical data associated with a procedure.

19. The apparatus of claim 17, wherein the processor is configured to determine if an alert should be generated based on at least one of situational data associated with a schedule of procedures and situational data associated with coordination of a plurality of procedures.

20. The apparatus of claim 17, wherein the processor is configured to determine if an alert should be generated based on priorities of each procedure from a plurality of procedures.

21. The apparatus of claim 17, wherein the processor is configured to determine if an alert should be generated based on prediction of a possible future condition of a patient based on a current condition of that patient.

22. The apparatus of claim 17, wherein the receiver is configured to receive a plurality of video signals of a plurality of patients from the plurality of monitors, each video signal from the plurality of video signals being uniquely associated with a monitor from the plurality of monitors and an associated patient from the plurality of patients, the display being configured to display at least one video signal from the received plurality of video signals.

23. The apparatus of claim 17, wherein the processor is further configured to allow a user to select a view from a plurality of views, the processor being configured to cause the display to display each view from the plurality of views when that view is selected by the user, each view from the plurality of views being associated with information associated with a patient, the processor being further configured to generate the alert associated with a view not selected by the user and to cause the view associated with the alert to be displayed.

24. The apparatus of claim 17, wherein the apparatus further comprises a portable processing device, the portable processing device including at least the receiver and the processor.

25. The apparatus of claim 17, wherein the apparatus further comprises a portable processing device, the portable processing device including the receiver, the processor, and the display, the display being configured to be viewed in a hands-free configuration by a user.

26. A processor-readable medium comprising code representing instructions to cause a processor to:
monitor data associated with a plurality of patients;
dynamically determine, based on at least one of historical information, situational information, and predictive information, if data associated with a patient from the plurality of patients are outside of a desired range; and
generate an alert in substantially real-time for data associated with a patient from the plurality of patients determined to be outside of the desired range.

27. The processor-readable medium of claim 26, wherein the code representing instructions to cause a processor to dynamically determine is based on at least one of historical information, situational information, and predictive information.

28. The processor-readable medium of claim 26, further comprising code representing instructions to cause a processor to:
display at least one of video telemetry and information substantially in real-time associated with each patient from the plurality of patients.

29. A processor-readable medium comprising code representing instructions to cause a processor to:
monitor data associated with a patient;
adaptively determine if an alert should be created for the patient at least partially based on patient-specific inputs;
if an alert is created, determine if the created alert should be generated at least partially based on a comparison of the created alert and a historical input; and
generate the created alert if it is determined that the created alert should be generated.

30. The processor-readable medium of claim 29, wherein the code representing instructions to cause a processor to determine if the created alert should be generated is further based on information about an example patient with a condition similar to a condition of the patient.

31. The processor-readable medium of claim 29, wherein the code representing instructions to cause a processor to adaptively determine if an alert should be created for the patient is further based on at least one of a physiologic parameter associated with the patient, a patient context associated with the patient, a procedure associated with the patient, a care process associated with the patient, and time parameters associated with the patient.
